(19) United States
(12) Patent Application Publication
(10) Pub. No.: US 2012/0202876 A1
(43) Pub. Date: Aug. 9, 2012

(54) METHODS OF TREATING DISEASES WITH PROANTHOCYANIDIN OLIGOMERS SUCH AS CROFELEMER

(75) Inventors: Alan S. Verkman, San Francisco, CA (US); Pravin Chaturvedi, San Francisco, CA (US); Lukmanee Tradtrantip, San Francisco, CA (US); Wan Namkung, San Francisco, CA (US)

(73) Assignees: NAPO PHARMACEUTICALS, INC., San Francisco, CA (US); REGENTS OF THE UNIVERSITY OF CALIFORNIA, Oakland, CA (US)

(21) Appl. No.: 13/500,589

(22) PCT Filed: Oct. 5, 2010
(86) PCT No.: PCT/US10/51530

§ 371 Date: Apr. 5, 2012

Related U.S. Application Data
(60) Provisional application No. 61/249,236, filed on Oct. 6, 2009.

(51) Int. Cl. A61K 31/353; A61P 43/00; A61P 11/00; A61P 21/00; A61P 9/00; A61P 7/00; C12N 5/071; A61P 25/00

(52) U.S. Cl. 514/456; 435/375

(57) ABSTRACT

Disclosed herein are methods of treating diseases including gastrointestinal disorders, such as secretory diarrheas, with the proanthocyanidin oligomer, crofelemer. Also disclosed is the anti-secretory effect of crofelemer on Calcium-activated chloride channels (CaCC) and Cystic fibrosis transmembrane conductance regulator (CFTR).

### CROSS-REFERENCE TO RELATED APPLICATIONS

This application claims priority under 35 U.S.C. §119(e) to U.S. Provisional Application No. 61/249,236 filed Oct. 6, 2009, which is hereby incorporated by reference in its entirety.

### FIELD OF THE INVENTION

The present embodiments relate to methods for the treatment of diseases comprising the administration of proanthocyanidin oligomers such as crofelemer. Some embodiments relate to the treatment of gastrointestinal disorders and other Cl⁻ channel associated diseases. Some embodiments relate to the treatment of inflammatory diseases. Other embodiments relate to the treatment of cancer. Some embodiments relate to methods of treating secretory diarrhea through inhibition of Cl⁻ channels.

### BACKGROUND

Inflammatory disease, cancer and bacteria-related diseases are still very difficult to treat effectively. For example, secretory diarrhea remains a global health challenge in developing and developed countries. Secretory diarrheas are characterized by loss of both fluid and electrolytes through the intestinal tract, leading to serious and often life-threatening dehydration. Secretory diarrheas are associated with a variety of bacterial, viral, and protozoal pathogens and can also result from other non-infectious etiologies such as ulcerative colitis, inflammatory bowel syndrome, and cancers and neoplasias of the gastrointestinal tract.

The sap of the South American medicinal plant, Croton lechleri (dragons blood), has been used in Ecuador and Peru for many years to treat diarrheas, including dysentery and cholera, as well as various lung, stomach and other conditions (Ulillas et al., 1994; Jones, 2003; Risco et al., 2003; Rossie et al., 2003). Crofelemer is purified from the blood-red bark latex of C. lechleri. Crofelemer is an amorphous, dark red-brown powder consisting of an oligomeric proanthocyanidin of varying chain lengths with an average molecular weight of 2100 daltons. Crofelemer has been characterized by ¹H-NMR, ¹³C-NMR, and mass spectrometry, producing the structure shown in FIG. 2A (Ubillas et al., 1994). The polymer chains of crofelemer range from 3 to 30 units and the monomeric components are (+)-catechin, (−)-epicatechin, (+)-gallocatechin, and (−)-epigallocatechin. Pharmacological studies have shown that crofelemer reduces fluid secretion in cell culture and mouse models (Gabriel et al., 1999).

### SUMMARY OF THE INVENTION

Some embodiments relate to a method of modulating Cl− secretion, comprising contacting a cell expressing calcium-activated chloride channel (CaCC) with an effective amount of crofelemer.

In some embodiments, the CaCC is TMEM16A.

Some embodiments relate to a method of modulating Cl− secretion, comprising contacting a cell expressing cystic fibrosis transmembrane conductance regulator (CFTR) with an effective amount of crofelemer.

Some embodiments relate to a method of modulating Na+ secretion, comprising contacting a cell expressing a Na+ channel with an effective amount of crofelemer.

In some embodiments, the Na+ channel is epithelial sodium channel (ENaC).

Some embodiments relate to a method of treating at least one gastrointestinal disorder, comprising inhibiting CaCC activity with an effective amount of crofelemer.

In some embodiments, the gastrointestinal disorder can be diarrhea, secretory diarrhea, irritable bowel syndrome, constipation, Crohn's disease, ulcers, anal fissures, constipation-predominant irritable bowel syndrome, diarrhea-predominant irritable bowel syndrome, alternating constipation-predominant/diarrhea-predominant irritable bowel syndrome, and abdominal discomfort associated with irritable bowel syndrome, diarrhea, secretory diarrhea, irritable bowel syndrome, constipation, Crohn's disease, ulcers, anal fissures, constipation-predominant irritable bowel syndrome, diarrhea-predominant irritable bowel syndrome, or alternating constipation-predominant/diarrhea-predominant irritable bowel syndrome.

In some embodiments, the gastrointestinal disorder is secretory diarrhea.

In some embodiments, the gastrointestinal disorder is irritable bowel syndrome.

Some embodiments relate to a method for treating at least one gastrointestinal disorder, comprising inhibiting CFTR activity with an effective amount of crofelemer.

In some embodiments, the gastrointestinal disorder can be diarrhea, secretory diarrhea, irritable bowel syndrome, constipation, Crohn's disease, ulcers, anal fissures, constipation-predominant irritable bowel syndrome, diarrhea-predominant irritable bowel syndrome, alternating constipation-predominant/diarrhea-predominant irritable bowel syndrome, and abdominal discomfort associated with irritable bowel syndrome, diarrhea, secretory diarrhea, irritable bowel syndrome, constipation, Crohn's disease, ulcers, anal fissures, constipation-predominant irritable bowel syndrome, diarrhea-predominant irritable bowel syndrome, or alternating constipation-predominant/diarrhea-predominant irritable bowel syndrome.

In some embodiments, the gastrointestinal disorder is secretory diarrhea.

In some embodiments, the gastrointestinal disorder is irritable bowel syndrome.

Some embodiments relate to a method of treating at least one channelopathy, comprising inhibiting CaCC activity with an effective amount of crofelemer.

In some embodiments, the channelpathy can be Cystic fibrosis, Erythromelalgia, Hyperkalemic periodic paralysis, Hypokalemic periodic paralysis, Long QT syndrome, Short QT syndrome, Malignant hyperthermia, Myotonia cogenita, and Neuromytonia.

Some embodiments relate to a method of treating at least one channelpathy, comprising inhibiting CFTR activity with an effective amount of crofelemer.

In some embodiments, the channelpathy is selected from a group consisting of Cystic fibrosis, Erythromelalgia, Hyperkalemic periodic paralysis, Hypokalemic periodic paralysis, Long QT syndrome, Short QT syndrome, Malignant hyperthermia, Myotonia cogenita, and Neuromytonia

Some embodiments relate to a method of treating at least one gastrointestinal disorder in a patient, comprising administering an effective amount of crofelemer to the patient.

In some embodiments, the gastrointestinal disorder can be diarrhea, secretory diarrhea, irritable bowel syndrome, constipation, Crohn's disease, ulcers, anal fissures, constipation-predominant irritable bowel syndrome, diarrhea-predominant irritable bowel syndrome, alternating constipation-predominant/diarrhea-predominant irritable bowel syndrome, and abdominal discomfort associated with irritable bowel syndromediarrhea, secretory diarrhea, irritable bowel syndrome, constipation, Crohn's disease, ulcers, anal fissures, constipation-predominant irritable bowel syndrome, diarrhea-predominant irritable bowel syndrome, or alternating constipation-predominant/diarrhea-predominant irritable bowel syndrome.

In some embodiments, the gastrointestinal disorder is secretory diarrhea.

In some embodiments, the gastrointestinal disorder is irritable bowel syndrome.

Some embodiments relate to a method for treating at least one cancer, comprising administering an effective amount of crofelemer to a patient.

In some embodiments, the cancer can be squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments, the cancer is colon cancer.

In some embodiments, the cancer is colorectal cancer.

Some embodiments relate to a method for treating at least one inflammatory disease, comprising administering an effective amount of crofelemer to a patient.

In some embodiments, the inflammatory disease can be Crohn's disease or irritable bowel syndrome.

In some embodiments, the inflammatory disease is irritable bowel syndrome.

Some embodiments relate to a method of treating at least one gastrointestinal disorder, comprising inhibiting ENaC activity with an effective amount of crofelemer.

In some embodiments, the gastrointestinal disorder can be diarrhea, secretory diarrhea, irritable bowel syndrome, constipation, Crohn's disease, ulcers, anal fissures, constipation-predominant irritable bowel syndrome, diarrhea-predominant irritable bowel syndrome, alternating constipation-predominant/diarrhea-predominant irritable bowel syndrome, and abdominal discomfort associated with irritable bowel syndrome, diarrhea, secretory diarrhea, irritable bowel syndrome, constipation, Crohn's disease, ulcers, anal fissures, constipation-predominant irritable bowel syndrome, diarrhea-predominant irritable bowel syndrome, or alternating constipation-predominant/diarrhea-predominant irritable bowel syndrome.

In some embodiments, the gastrointestinal disorder is secretory diarrhea.

In some embodiments, the gastrointestinal disorder is irritable bowel syndrome.

### BRIEF DESCRIPTION OF THE DRAWINGS

FIG. 1 shows chloride and intestinal fluid secretion through apical membrane chloride channels of enterocytes.

FIG. 2A shows the chemical structure of crofelemer, which consists of a mixture of proanthocyanidin oligomers. FIG. 2B shows graphs which indicate that crofelemer reduces Cl⁻ secretion in T84 human intestinal cells in response to cAMP and calcium-elevating agonists. FIG. 2B also shows short-circuit current in T84 cells following activation of Cl⁻ secretion by forskolin (10 μM), ATP (100 μM) or thapsigargin (1 μM). Indicated concentrations of crofelemer were added to the luminal bathing solution. Where indicated, cells were pre-treated with 20 μM CFTRᵢₙₕ-172 to inhibit CFTR Cl⁻ current.

FIG. 3 shows graphs indicating crofelemer inhibition of CFTR Cl⁻ conductance. FIG. 3A shows apical membrane current in CFTR-expressing FRT cells following permeabilization with amphotericin B and in the presence of a transepithelial Cl⁻ gradient (apical [Cl⁻] 75 mM, basolateral [Cl⁻] 150 mM). CFTR Cl⁻ conductance was activated by 100 μM CPT-cAMP followed by addition of indicated concentrations of crofelemer to the luminal solution. FIG. 3B shows crofelemer concentration-inhibition of CFTR Cl⁻ current measured at 20 min after crofelemer application (S.E. n=3-5). Data shown for experiments as in A (open circles) and with reversed Cl⁻ gradient (apical [Cl⁻] 150 mM, basolateral [Cl⁻] 75 mM) (filled circles).

FIG. 4 shows graphs characterizing crofelemer's inhibition of CFTR Cl⁻ conductance. FIG. 4A shows crofelemer inhibition of CFTR following different agonists including genistein (50 μM), forskolin (20 μM) and IBMX (100 μM). FIG. 4B shows the slow reversibility of crofelemer inhibition of CFTR. Where indicated, crofelemer was added, the apical solution was washed extensively, and CPT-cAMP re-added. FIG. 4C shows inhibition of CFTR by the small-molecule CFTR inhibitors, CFTRᵢₙₕ-172 or GlyH-101, in the absence or presence of crofelemer pre-treatment. (left) Apical membrane current following CFTR activation by CPT-cAMP and inhibition by CFTRᵢₙₕ-172 or GlyH-101. (right) Crofelemer (50 μM) was added to inhibit CFTR Cl⁻ current by ˜50-60%, followed by indicated concentrations of CFTRᵢₙₕ-172 or GlyH-101.

FIG. 5 shows graphs depicting the results of patch-clamp analysis of crofelemer inhibition of CFTR. (left) Whole-cell CFTR current recorded at a holding potential at 0 mV, and pulsing to voltages between ±100 mV in steps of 20 mV in the absence and presence of 50 μM crofelemer. CFTR was stimulated by forskolin. (right) Current/voltage (I/V) plot of mean currents at the middle of each voltage pulse from experiments as in A (S.E., n=3). Fitted IC₅₀ 6.5 μM.

FIG. 6 shows graphs depicting Crofelemer inhibition of calcium-activated Cl⁻ channels. FIG. 6A shows apical membrane current in TMEM16A-expressing FRT cells in the presence of a transepithelial Cl⁻ gradient (apical [Cl⁺] 70 mM, basolateral [Cl⁻] 140 mM). FIG. 6B shows crofelemer concentration-dependence of TMEM16A Cl⁻ current inhibition. FIG. 6C shows whole-cell TMEM16A current recorded at a holding potential at 0 mV, and pulsing to voltages between ±100 mV in steps of 20 mV in the absence and presence of 10 μM Crofelemer. TMEM16A was stimulated by 100 μM ATP. FIG. 6D shows a Current/voltage (I/V) plot of mean currents (at the middle of each voltage pulse).

FIG. 7 shows graphs indicating that that crofelemer has little or no effect on apical membrane cation channels and intracellular cAMP and calcium signaling. FIG. 7A (left) shows short-circuit current in primary cultures of CFTR-deficient human bronchial epithelial cells without vs. with pre-treatment with 50 μM crofelemer in the luminal solution. Where indicated, amiloride (10 μM) and UTP (100 μM) were added. FIG. 7A (right) shows a summary of differences in short-circuit current following amiloride and UTP additions (S.E., n=3, * P<0.05). FIG. 7B shows apical membrane K⁺ current in human bronchial epithelial cells following basolateral membrane permeabilization with 20 μM amphotericin B and in the presence of a K⁺ gradient (apical [K⁺] 5 mM, basolateral [K⁺] 150 mM). FIG. 7C shows cyclic AMP levels in T84 cell homogenates under basal conditions and at 10 min after treatment with 20 μM forskolin. Differences +/− crofelemer not significant. FIG. 7D shows calcium signaling measured by fura-2 fluorescence in T84 cells under basal conditions and following ATP (100 μM). Where indicated cells were pre-treated with 50 μM crofelemer. Inset summarizes the peak ATP increase in fura-2 fluorescence ratio (S.E., n=4). The difference between the control and crofelemer is not significant.

### DETAILED DESCRIPTION OF THE INVENTION

The present embodiments relate to treatment of a wide variety of diseases, medical conditions and disorders including for example, inflammatory diseases, neoplastic diseases, bacteria related diseases, viral related diseases, channelopathies, gastrointestinal disorders and infertility. Examples of channelopathies include, but are not limited to Cystic fibrosis, Erythromelalgia, Hyperkalemic periodic paralysis, Hypokalemic periodic paralysis, Long QT syndrome, Short QT syndrome, Malignant hyperthermia, Myotonia cogenita, and Neuromytonia. Examples of cancer include but are not limited to bone cancer, lung cancer, skin cancer, colorectal cancer, familial adenomatous polyposis and retinoblastoma. Examples of gastrointestinal disorders include but are not limited to diarrhea, secretory diarrhea, irritable bowel syndrome, constipation, Crohn's disease, ulcers, anal fissures, constipation-predominant irritable bowel syndrome, diarrhea-predominant irritable bowel syndrome, alternating constipation-predominant/diarrhea-predominant irritable bowel syndrome and abdominal discomfort associated with any of the above gastrointestinal disorders.

The present embodiments also relate to the treatment of diseases including, but not limited to cachexia, cardiovascular disease, immune disease, tuberculous pleurisy, rheumatoid pleurisy, fatigue associated with cancer or its treatment, cardiovascular disease, skin redness, diabetes, transplant rejection, otitis media (inner ear infection), sinusitis and viral infection, septic shock, transplantation, graft-vs-host disease, ischemia/reperfusion injury, Graves' ophthalmopathy, Hashimoto's thyroiditis, thryoid-associated ophthalmopathy, nodular goiter, herpetic stromal keratitis, microbial keratitis, peripheral ulcerative keratitis, Behcet's disease, uveitis, vitreoretinal proliferative disease, rabies virus ocular disease, Vogt-Koyanagi-Harada's disease, retinopathy, retinal laser photocoagulation, acute retinal necrosis syndrome, systemic vasculitis, recurrent aphthous stomatitis, neovascular glaucoma, eye infections, ocular allergic diseases, retinal detachment, optic neuritis, multiple sclerosis, systemic sclerosis, hereditary retinal degeneration, trachoma, autoimmune diseases, chemotherapy related mucosal injury, affective disorders, including depressive disorders (major depressive disorder, dysthymia, childhood depression, atypical depression, bipolar disorder, mania and hypomania) and anxiety disorders (generalized anxiety disorder, social anxiety disorder, phobias, obsessive compulsive disorder, panic disorder, post-traumatic stress disorder); premenstrual dysphoric disorder (also known as pre-menstrual syndrome); psychotic disorders, such as brief psychotic disorder, schizophrenia, psychotic mood disorder (depression and/or mania); attention deficit disorder (with and without hyperactivity); obesity, eating disorders such as anorexia nervosa and bulimia nervosa; vasomotor flushing; cocaine and alcohol addiction, sexual dysfunction and related illnesses; acute and chronic pain syndromes, as exemplified by fibromyalgia, arthritis, chronic low back pain, trigeminal neuralgia; visceral pain syndromes, such as irritable bowel syndrome, noncardiac chest pain, functional dyspepsia, interstitial cystitis, essential vulvodynia, urethral syndrome, orchialgia, temperomandibular disorder, atypical face pain, migraine headache, and tension headache; functional somatic disorders, for example, chronic fatigue syndrome; neurologic disorders including seizure disorder, Tourette Syndrome, Parkinson's Disease, Huntington's Chorea, Alzheimer's Disease, subcortical and other dementias, Tardive Dyskinesia, Multiple Sclerosis, Rett Syndrome or amyotrophic lateral sclerosis restenosis, asthma, chronic obstructive lung diseases, abnormal angiogenesis, carcinoma, lymphoma, blastoma, sarcoma, and leukemia or lymphoid malignancies. Other examples of cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Some embodiments relate to the treatment of secretory diarrhea, irritable bowel syndrome and colon cancer.

Secretory diarrheas can be characterized by the loss of both fluid and electrolytes through the intestinal tract, leading to serious and often life-threatening dehydration. Secretory diarrheas are associated with a variety of bacterial, viral, and protozoal pathogens and may also result from other non-infectious etiologies such as ulcerative colitis, inflammatory bowel syndrome, and cancers and neoplasias of the gastrointestinal tract.

Two major bacterial sources of secretory diarrhea are Vibrio cholerae and Escherichia coli. The enterotoxigenic types of E. coli represent an important source of secretory diarrhea in developing countries and are associated with secretory diarrhea. Other strains of E. coli which cause diarrhea include enterohemorrhagic, enteroinvasive, and enteropathogenic and other strains. Other bacterial agents associated with secretory diarrhea include other Vibrio spp., Campylobacter spp., Salmonella spp., Aeromonas spp., Plesiomonas spp., Shigella spp., Klebsiella spp., Citrobacter spp., Yersinia spp., Clostridium spp., Bacteriodes spp., Staphylococcus spp., and Bacillus spp, as well as other enteric bacteria.

Secretory diarrhea can also be associated with protozoal pathogens such as Cryptosporidium spp, for example Cryptosporidium parvum. See generally, Holland, 1990, Clin. Microbiol. Rev. 3:345; Harris, 1988, Ann. Clin. Lab. Sci. 18:102; Gracey, 1986, Clin. in Gastroent., 15:21; Ooms and Degryse, 1986, Veterinary Res. Comm. 10:355; Black, 1982, Med. Clin. Nor. Am., 66:611.

Secretory diarrheas can also be associated with viral infections, such as, diarrheas which accompany Human Immunodeficiency Virus (HIV) infection and Acquired Immuno Deficiency Syndrome (AIDS), and rotavirus infection, in particular. Almost all AIDS patients suffer from diarrhea at some point during the course of the disease, and 30% of AIDS patients suffer from chronic diarrhea. The diarrhea that accompanies AIDS has been termed “HIV-Associated Chronic Diarrhea.” This diarrheal component of HIV disease is thought to be associated with, at least in some patients, by a secondary infection of protozoal pathogens, for example Cryptosporidium spp. Additionally, rotavirus infection is associated with diarrhea for example in infants and young children in developing countries.

Secretory diarrhea is also a problem in non-human animals, for example in farm animals, such as bovine animals, swine, sheep (ovine animals), poultry (such as chickens), and equine animals, and other domesticated animals such as canine animals and feline animals. Diarrheal disease is seen in young and recently weaned farm animals. Diarrheal disease in farm animals, for example food animals such as cattle, sheep and swine, is often associated with bacterial pathogens such as enterotoxigenic, enterohemorrhagic and other E. coli, Salmonella spp., Clostridium perfringens, Bacteriodes fragilis, Campylobacter spp., and Yersinia enterocolitica. Additionally, protozoal pathogens, for example Cryptosporidium parvum, and viral agents, for example rotaviruses and coronaviruses, are associated with diarrhea in farm animals. Examples of other viral agents which have been implicated in diarrhea of farm animals include togavirus, parvovirus, calicivirus, adenoviruses, bredaviruses, and astroviruses. See generally Holland, 1990, Clin. Microbiology Rev. 3:345; see also Gutzwiller and Blum, 1996, AJVR 57:560; Strombeck, 1995, Veterinary Quarterly 17(Suppl. 1):S12; Vermunt, 1994, Austral. Veterinary J. 71:33; Driesen et al., 1993, Austral. Veterinary J. 70:259; Mouricout, 1991, Eur. J. Epidemiol. 7:588; Ooms and Degryse, 1986, Veterinary Res. Comm. 10:355.

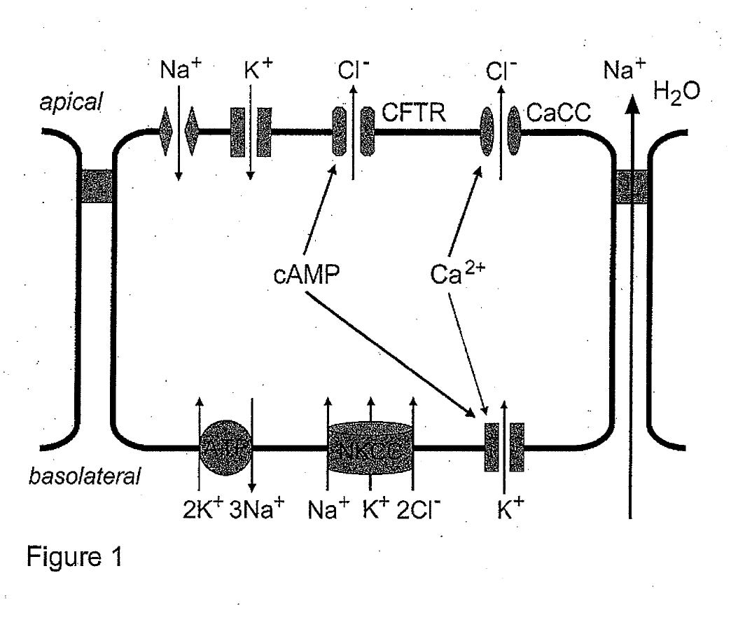

Secretory diarrheal disorders of various etiologies share the common feature of excessive Cl⁻ secretion. Intestinal fluid secretion involves Cl⁻ influx into enterocytes though a Na⁺/K⁺/2Cl⁻ symporter on the basolateral membrane, and Cl⁻ efflux through apical (lumen-facing) Cl⁻ channels (Barrett and Keely, 2000; Field, 2003; Thiagarajah and Verkman, 2005) (FIG. 1). Not wishing to be bound to a particular theory, K⁺ channels and a 3Na⁺/2K⁺ pump establish the electrochemical driving force for Cl⁻ secretion. Na⁺ and water secretion follow passively in response to active Cl⁻ secretion. Bacterial enterotoxins, such as those produced by Vibrio choleraeand Escherichia coli, elevate cyclic nucleotide concentrations in enterocytes, resulting in Cl⁻ channel activation and fluid secretion. Na⁺ absorption through apical membrane Na⁺ channels and electrogenic Na⁺-coupled symporters oppose net fluid secretion. The rate of net intestinal fluid secretion, and hence the severity of secretory diarrhea, is associated with modulators of these transporting systems and to upstream cyclic nucleotide or calcium signaling pathways.

Some embodiments relate to the cellular antisecretory targets of crofelemer. Some embodiments relate to the principal luminal membrane determinants of intestinal fluid secretion, including, for example, ion channels and signaling pathways. Some embodiments relate to the use of crofelemer to inhibit apical membrane cAMP-stimulated (CFTR) and calcium-stimulated (CaCC) channels, with little effect on cation channels or cAMP/calcium signaling. In some embodiments, crofelemer inhibits two distinct Cl⁻ channels, which are unrelated in their sequences and structures. Without wishing to be bound to a particular theory, the ability of crofelemer to inhibit Cl⁻ channels in addition to its slow washout appears to provide its broad antisecretory activity in diarrheas associated with bacterial enterotoxins, viruses and other effectors. In some embodiments, the inhibition of both CFTR and CaCCs is useful due to cAMP/calcium cross-talk in enterocytes and the involvement of two types of Cl⁻ channels in some diarrheas.

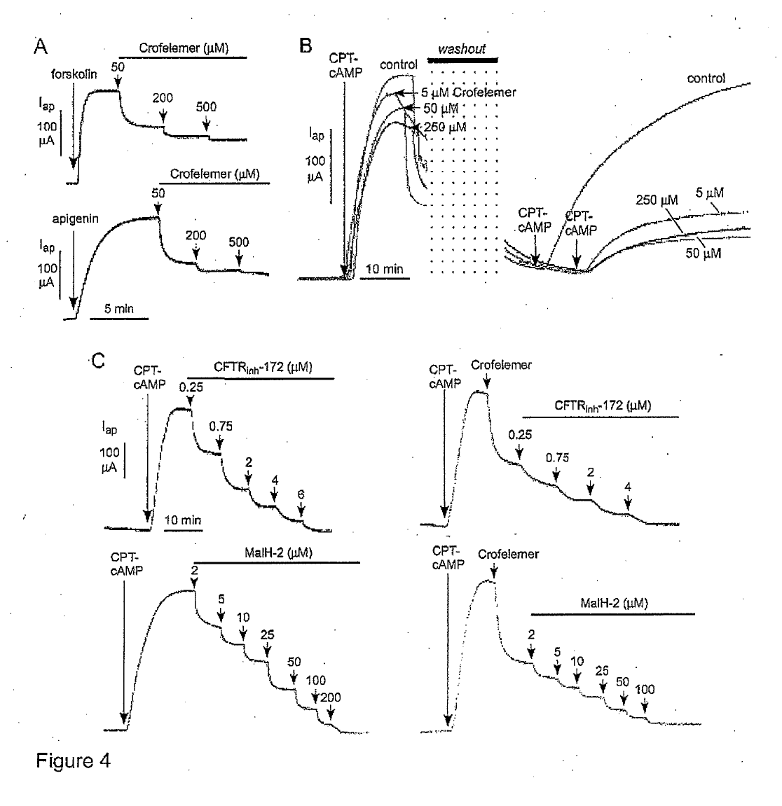

Some embodiments relate to the use of crofelemer as a partial antagonist of CFTR Cl⁻ conductance, with a concentration-dependent rate of inhibition over several minutes. Washout of the crofelemer is slow, occurring over several hours. Unlike thiazolidinone and glycine hydrazide CFTR inhibitors, crofelemer inhibition of CFTR Cl⁻ conductance is partial even at high concentrations. Some embodiments relate to partial external CFTR pore blockade by the crofelemer molecule and/or an intrinsically inefficient allosteric inhibition mechanism which is associated with the partial inhibition. In one embodiment, patch-clamp analysis was used to determine that crofelemer action on the extracellular-facing CFTR surface which produces voltage-independent channel inhibition without direct pore occlusion. In contrast, CFTR inhibitors of the glycine hydrazide class produced a voltage-dependent block, with inward rectification of residual CFTR Cl⁻ current, and direct pore occlusion with rapid flicker in membrane current (Muanprasat et al., 2004; Sonawane et al., 2006, 2007, 2008). The independence of crofelemer and GlyH-101 action seen in FIGS. 4C and D is consistent with crofelemer action at site different from that of GlyH-101, which occludes the CFTR pore. The larger molecular size of crofelemer compared to GlyH-101 is consistent with crofelemer action at a site outside of the CFTR pore. Prior studies (Gabriel et al., 1999) provide evidence for CFTR inhibition by crofelemer in T84 cells in the presence of a large Cl⁻ gradient.

In some embodiments, crofelemer strongly inhibits CaCC(s). CaCCs in intestinal epithelial cells provide an important route for Cl⁻ and fluid secretion in secretory diarrheas associated with certain drugs, including some antiretrovirals and chemotherapeutics, and some viruses (Morris et al. 1999, Barrett, 2000; Kidd and Thorn, 2000; Takahashi et al. 2000; Gyomorey et al. 2001; Rufo et al. 2004; Thiagarajah and Verkman, 2005; Schultheiss et al. 2005, 2006; Farthing, 2006; Lorrot and Vasseur, 2007).

In addition to their expression in intestinal epithelial cells, CaCCs are broadly expressed in many cell types where they are involved in different functions, including, but not limited to transepithelial fluid secretion, olfactory and sensory signal transduction, smooth muscle contraction, and cardiac excitation (Hartzell et al., 2005; Verkman and Galietta, 2009). The molecular identity of CaCCs has been enhanced by the finding that TMEM16A (anoctamin-1) is a CaCC (Caputo et al., 2008; Schroeder et al., 2008; Yang et al., 2008). Several lines of evidence support the conclusion that TMEM16A is a CaCC. For example, it has been demonstrated that CaCC Cl⁻ currents in TMEM16A-transfected cells are similar in electrophysiological characteristics with native CaCCs, and that CaCC Cl⁻ current is reduced following RNAi knockdown of TMEM16A. TMEM16A is expressed broadly in epithelial and other cell types in multiple organs, including, for example, intestinal epithelium.

In some embodiments, crofelemer is used to inhibit human TMEM16A. Not wishing to be bound to a particular theory, inhibition of TMEM16A by crofelemer is associated with its inhibition of Cl⁻ current in T84 cells following addition of calcium-elevating agonists. In some embodiments, crofelemer was found to strongly inhibit the intestinal calcium-activated Cl⁻ channel TMEM16A with maximum inhibition >90% and IC₅₀ of 6.5 μM, and a voltage-independent inhibition mechanism. As CaCCs are broadly expressed in many cell types in addition to their expression in intestinal epithelial cells, in some embodiments crofelemer's inhibitory effect on CaCC can be utilized to modulate influx and efflux of Cl⁻ in a wide variety applications where CaCCs are involved in different functions, including, but not limited to transepithelial fluid secretion, olfactory and sensory signal transduction, smooth muscle contraction, and cardiac excitation (Hartzell et al., 2005; Verkman and Galietta, 2009).

In some embodiments, the cellular antisecretory action of crofelemer involves two distinct Cl⁻ channel targets on the luminal membrane of epithelial cells lining the intestine, providing dual inhibition of CFTR and CaCC Cl⁻ channels.

Some embodiments relate to targeted inhibitors of membrane Cl⁻ channels, the cystic fibrosis transmembrane regulator conductance (CFTR), a cAMP-stimulated Cl⁻ channel, and calcium-activated Cl⁻ channels (CaCCs). In some embodiments high-throughput screening and follow-up chemistry, can identify inhibitors of these Cl⁻ channels, for example, nanomolar-potency thiazolidinone (Ma et al., 2002) and glycine hydrazide (Muanprasat et al., 2004) CFTR inhibitors, and 3-acyl-2-aminothiophene CaCC inhibitors (de la Fuente et al., 2008). Thiophenecarboxylate activators of phosphodiesterases that reduce cyclic nucleotide concentrations and toxin-induced intestinal fluid secretion have also been identified (Tradtrantip et al., 2008).

Crofelemer reduces chloride flux across intestinal epithelial cells and reduces fluid movement into the intestinal lumen which results in fluid loss and dehydration associated with secretory diarrhea. Thus, pharmaceutical formulations containing crofelemer or other inhibitors of CFTR and/or CaCC Cl⁻ channels are useful in prophylactic and therapeutic applications against secretory diarrhea, for example in preventing the dehydration and electrolyte loss that accompanies secretory diarrhea. In other embodiments, pharmaceutical formulations containing crofelemer or other inhibitors of CFTR and/or CaCC Cl⁻ channels are useful in prophylactic and therapeutic applications against diseases involving abnormal Cl⁻ influx and efflux.

The pharmaceutical formulations containing crofelemer or other inhibitors of CFTR and/or CaCC Cl⁻ channels can be used therapeutically or prophylactically against any type of secretory diarrhea in either humans or animals. In a preferred embodiment, the pharmaceutical formulation containing crofelemer or other inhibitors of CFTR and/or CaCC Cl⁻ channels is used to treat secretory diarrheas associated with enteric bacteria. These enteric bacteria include, but are not limited to, Vibrio cholerae, E. coli, including the enteropathogenic, enterotoxigenic, enteroadherent, enterohemorrhagic, or enteroinvasive types of E. coli, other Vibrio spp., Campylobacter spp., Salmonella spp., Aeromonas spp., Plesiomonas spp., Shigella spp., Klebsiella spp., Citrobacter spp., Yersinia spp., Clostridium spp., Bacteriodes spp., Staphylococcus spp., and Bacillus spp. This embodiment also includes the treatment of traveler's diarrhea.

In another embodiment, the pharmaceutical formulation containing crofelemer or other inhibitors of CFTR and/or CaCC Cl⁻ channels is used to treat secretory diarrhea associated with protozoa, including but not limited to, Giardia and Cryptosporidium spp., for example Cryptosporidium parvum.

In another embodiment, the pharmaceutical formulation containing crofelemer or other inhibitors of CFTR and/or CaCC Cl⁻ channels is used to treat secretory diarrhea associated with non-infectious etiologies, such as but not limited to, non-specific diarrhea, inflammatory bowel syndrome, ulcerative colitis, and cancers and neoplasias of the gastrointestinal tract.

In another embodiment, the pharmaceutical formulations containing crofelemer or other inhibitors of CFTR and/or CaCC Cl⁻ channels are used for the treatment of HIV-Associated Chronic Diarrhea in patients with AIDS. In yet another embodiment, the pharmaceutical formulation is used to treat diarrhea in infants or children, including but not limited to, diarrhea associated with rotavirus.

In another embodiment, the pharmaceutical formulations containing crofelemer or other inhibitors of CFTR and/or CaCC Cl⁻ channels are used for treating and/or preventing one or more symptoms associated with constipation-predominant irritable bowel syndrome (c-IBS), in warm blooded animals, including male and female humans, which symptoms include, but are not limited to, pain, abdominal discomfort and abnormal stool frequency. The methods of the invention generally comprise administering to a subject in need of c-IBS treatment a pharmaceutical formulations containing crofelemer or other inhibitors of CFTR and/or CaCC Cl⁻ channels.

In another embodiment, the pharmaceutical formulations containing crofelemer or other inhibitors of CFTR and/or CaCC Cl⁻ channels are used for providing a method of treating pain associated with c-IBS comprising administering to a patient in need of such treatment, an amount of a pharmaceutical formulations containing crofelemer or other inhibitors of CFTR and/or CaCC Cl⁻ channels effective to treat pain associated with c-IBS.

The pharmaceutical formulations containing crofelemer or other inhibitors of CFTR and/or CaCC Cl⁻ channels can also be used to treat diarrhea in non-human animals, for example in farm animals, such as but not limited to, bovine animals, swine, ovine animals, poultry (such as chickens), and equine animals, and other domesticated animals such as canine animals and feline animals. In particular the pharmaceutical formulations of the invention can be used to treat diarrheal disease in non-human animals, for example food animals such as cattle, sheep and swine, associated with bacterial pathogens such as enterotoxigenic, enterohemorrhagic and other E. coli, Salmonella spp., Clostridium perfringens, Bacteriodes fragilis, Campylobacter spp., and Yersinia enterocolitica, protozoal pathogens, for example Cryptosporidium parvum, and viral agents, for example rotaviruses and coronaviruses, but also togavirus, parvovirus, calicivirus, adenoviruses, bredaviruses, and astroviruses.

Additionally, the pharmaceutical formulations containing crofelemer or other inhibitors of CFTR and/or CaCC Cl⁻ channels may also be administered prophylactically to humans and non-human animals to prevent the development of secretory diarrhea.

The pharmaceutical compositions containing crofelemer or other inhibitors of CFTR and/or CaCC Cl⁻ channels can be administered to AIDS patients to prevent the occurrence of HIV-Associated Chronic Diarrhea. Also, the pharmaceutical compositions containing crofelemer or other inhibitors of CFTR and/or CaCC Cl⁻ channels can be administered to children in a community threatened with cholera epidemic or rotavirus epidemic to prevent the spread of the disease. Likewise, the pharmaceutical compositions containing crofelemer or other inhibitors of CFTR and/or CaCC Cl⁻ channels of can be administered to farm animals, for example young or recently weaned farm animals, to prevent the development of diarrheal disease.

The pharmaceutical formulations can also be administered either alone or in combination with other agents for treatment or amelioration of secretory diarrhea symptoms such as rehydration agents, antibiotics, anti-motility agents, and fluid adsorbents, such as attapulgite.

The pharmaceutical formulations containing crofelemer or other inhibitors of CFTR and/or CaCC Cl⁻ channels can also be incorporated into animal feed for use in treating secretory diarrhea in animals such as bovine animals, swine, ovine animals, poultry, equine animals, canine animals, and feline animals.

It is to be understood that both the foregoing general description and the following detailed description are exemplary and explanatory only and are not restrictive of the invention as claimed. In this application, the use of the singular includes the plural unless specifically stated otherwise.

In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

All documents, or portions of documents, cited in this application, including but not limited to patents, patent applications, articles, books, and treatises, are hereby expressly incorporated by reference in their entirety for any purpose.

As will be readily apparent to one skilled in the art, the useful in vivo dosage to be administered and the particular mode of administration will vary depending upon the age, weight, medical condition of the patient, the severity of the condition to be treated, the route of administration, the renal and hepatic function of the patient, and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Advantageously, compounds of the present embodiments may be administered, for example, in a single daily dose, or the total daily dosage may be administered in divided doses of two, three, or four times daily.

The daily dosage of the products may be varied over a wide range; e.g., from about 0.5 to about 10,000 mg per adult human per day. For oral administration, the formulations are preferably provided in the form of tablets containing about 0.5, 1.0, 2.0, 3.0, 4.0, 5.0, 6.0, 7.0, 8.0, 9.0, 10.0, 15.0, 25.0, 50.0, 100, 200, 300, 400, 500, 600, 700, 800, 900, 1000, 2000, 3000, 4000, 5000, 6000, 7000, 8000, 9000 or 10,000 milligrams of the active ingredient for the symptomatic adjustment of the dosage to the patient to be treated. The instant pharmaceutical formulations typically contain from 10 mg to about 2000 mg of the instant compounds, preferably, from about 50 mg to about 1000 mg of active ingredient. An effective amount of the instant compounds is ordinarily supplied at a dosage level of from about 0.002 mg/kg to about 150mg/kg of body weight per day. Preferably, the range is from about 0.02 to about 80 mg/kg of body weight per day, and especially from about 0.2 mg/kg to about 40 mg/kg of body weight per day. The compounds may be administered on a regimen of about 1 to about 10 times per day.

In some embodiments the oral dose of crofelemer is 100 mg, 125 mg, 250 mg, 300 mg, 500 mg, or 1,000 mg. In several embodiments an oral dose of crofelemer is administered twice daily. In other embodiments, an oral dose of crofelemer is administered once daily. In several embodiments a patient is administered daily dose of crofelemer for a period of about one day, two days, seven days, 14 days, 28 days, 60 days, or more than 90 days.

As used herein, an “increase” or “decrease” in a measurement, unless otherwise specified, is typically in comparison to a baseline value. For example, an increase in time to hospitalization for subjects undergoing treatment may be in comparison to a baseline value of time to hospitalization for subjects that are not undergoing such treatment. In some instances an increase or decrease in a measurement can be evaluated based on the context in which the term is used.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The term “effective amount” includes an amount effective, at dosages and for periods of time necessary, to achieve the desired result, e.g., sufficient to treat or gastrointestinal disorders in a patient or subject. An effective amount of crofelemer may vary according to factors such as the disease state, age, and weight of the subject, and the ability of crofelemer to elicit a desired response in the subject. Dosage regimens may be adjusted to provide the optimum therapeutic response. An effective amount is also one in which any toxic or detrimental effects (e.g., side effects) of crofelemer are outweighed by the therapeutically beneficial effects.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with crofelemer, where the untreated subjects have, or are subject to developing, the same or similar disease, condition, symptom, or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of gastrointestinal disorders in a subject. Amelioration may be transient, prolonged or permanent or it may be variable at relevant times during or after crofelemer is administered to a subject or is used in an assay or other method described herein or a cited reference, e.g., within timeframes described infra, or about 1 hour after the administration or use of crofelemer to about 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with crofelemer, where the untreated subjects have, or are subject to developing, the same or similar disease, condition, symptom, or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after crofelemer is administered to a subject or is used in an assay or other method described herein or a cited reference, e.g., within times descried infra, or about 1 hour of the administration or use of crofelemer to about 3, 6, 9 months or more after a subject(s) has received crofelemer.

The term “modulate” may also refer to increases or decreases in the activity of a cell in response to exposure to crofelemer, e.g., the inhibition of proliferation and/or induction of differentiation of at least a sub-population of cells in an animal such that a desired end result is achieved, e.g., a therapeutic result of crofelemer used for treatment may increase or decrease over the course of a particular treatment.

The term “obtaining” as in “obtaining crofelemer” is intended to include purchasing, synthesizing or otherwise acquiring crofelemer.

The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The language “a prophylactically effective amount” of a compound refers to an amount of crofelemer which is effective, upon single or multiple dose administration to the subject, in preventing or treating gastrointestinal disorders.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

The compositions may be in the form of tablets, capsules, powders, granules, lozenges, liquid, gel preparations, sterile parenteral solutions or suspensions, metered aerosol or liquid sprays, drops, ampoules, auto-injector devices or suppositories; for oral, parenteral, intranasal, sublingual, buccal, topical or rectal administration, or for administration by inhalation or insufflation. Tablets and capsules for oral administration may be in a form suitable for unit dose presentation and may contain conventional excipients. Examples of these are: binding agents such as syrup, acacia, gelatin, sorbitol, tragacanth, and polyvinylpyrrolidone; fillers such as lactose, sugar, maize-starch, calcium phosphate, sorbitol or glycine; tableting lubricants, such as magnesium stearate, silicon dioxide, talc, polyethylene glycol or silica; disintegrants, such as potato starch; or acceptable wetting agents, such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents.

For oral administration, crofelemer can be formulated readily by combining crofelemer with pharmaceutically acceptable carriers well known in the art. Such pharmaceutically acceptable carriers enable the compounds of the present embodiments to be formulated as tablets, pills, dragees, capsules, liquids, gels, syrups, slurries, suspensions and the like, for oral ingestion by a patient to be treated. Pharmaceutical formulations for oral use can be obtained by combining crofelemer with solid excipient, optionally grinding a resulting mixture, and processing the mixture of granules, after adding suitable auxiliaries, if desired, to obtain tablets or dragee cores. If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures.

The phrases “systemic administration,” “administered systemically,” “peripheral administration,” and “administered peripherally,” as used herein mean the administration of crofelemer such that it enters the subject's system and, thus, is subject to metabolism and other like processes, for example, subcutaneous administration.

The language “therapeutically effective amount” of crofelemer refers to an amount of a crofelemer which is effective, upon single or multiple dose administration to the subject, in inhibiting the bacterial growth and/or invasion, or in decreasing symptoms, such as gastrointestinal disorders such as diarrhea. “Therapeutically effective amount” also refers to the amount of a therapy (e.g., a composition comprising crofelemer), which is sufficient to reduce the severity of a gastrointestinal disorder in a subject.

As used herein, the terms “prevent,” “preventing,” and “prevention” refer to the prevention of the recurrence, onset, or development of gastrointestinal disorder episodes. Preventing includes protecting against the occurrence and severity of gastrointestinal disorder episodes.

As used herein, the term “prophylactically effective amount” refers to the amount of a therapy (e.g., a composition comprising crofelemer) which is sufficient to result in the prevention of the development, recurrence, or onset of gastrointestinal disorder episodes or to enhance or improve the prophylactic effect(s) of another therapy.

As used herein, “subject” includes organisms which are capable of suffering from a gastrointestinal disorder or other disorder treatable by crofelemer or who could otherwise benefit from the administration of crofelemer as described herein, such as human and non-human animals. Preferred human animals include human subjects. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The following Examples are presented for the purposes of illustration and should not be construed as limitations.

### EXAMPLE 1

### Crofelemer Inhibits Secretion By T84 Human Intestinal Epithelial Cells

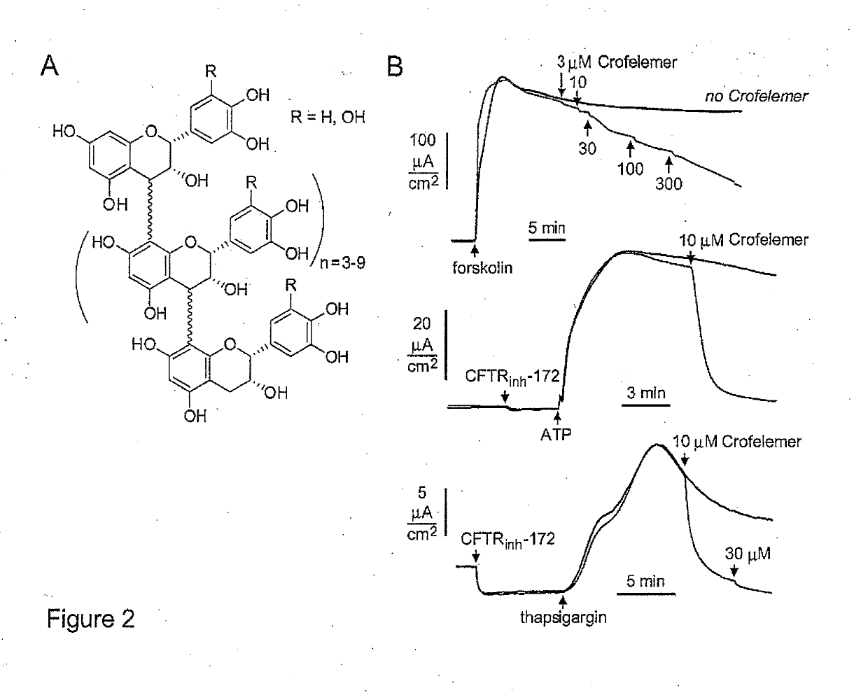

To test whether crofelemer reduces intestinal cell Cl⁻ secretion, short-circuit current was measured in T84 cells in symmetrical physiological solutions (without plasma membrane permeabilization). FIG. 2B shows crofelemer concentration-dependent inhibition of the increase in short-circuit current produced by the cAMP agonist forskolin (top) and the calcium agonists ATP (middle) and thapsigargin (bottom). Measurements with the calcium agonists were done in the presence of CFTRᵢₙₕ-172 to inhibit CFTR. Whereas crofelemer inhibition of forskolin-induced current, which is mainly CFTR-dependent, was slow, weak and partial—inhibition of ATP and thapsigargin-induced current was nearly complete at 10 μM crofelemer. Not wishing to be bound to a particular theory, this inhibition of current induced by calcium agonists suggests that crofelemer inhibits both CFTR and CaCC channels, with apparently much stronger inhibition of the latter. Further measurements were done using transfected cell systems to study crofelemer effects on CFTR and CaCCs in isolation.

### EXAMPLE 2

### Crofelemer is a Partial Antagonist of CFTR Cl⁻ Conductance

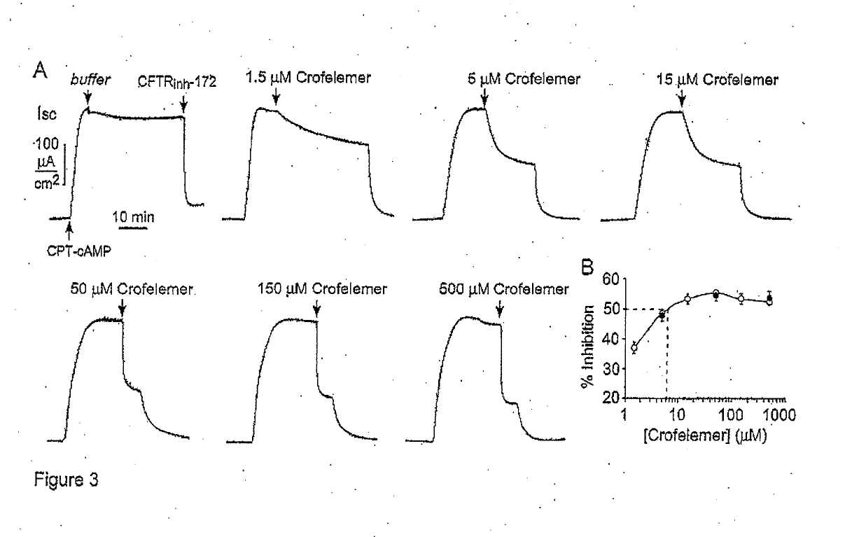

CFTR Cl⁻ current was measured in CFTR-expressing FRT cells in which the basolateral membrane was permeabilized by amphotericin B and a transepithelial Cl⁻ gradient was applied. Under these conditions, the measured current provides a direct quantitative measure of CFTR Cl⁻ conductance. FIG. 3A shows apical membrane current measurements in which CFTR Cl⁻ conductance was stimulated by CPT-cAMP and followed by addition of different concentrations of crofelemer in the apical bathing solution. Increasing concentrations of crofelemer produced notably more rapid, though partial, inhibition of CFTR Cl⁻ current. Addition of crofelemer to the basolateral bathing solution did not inhibit current (not shown). As summarized in FIG. 3B (open circles) the apparent IC₅₀ (giving 50% inhibition of Cl⁻ current) for crofelemer was ˜7 μM, and the maximal inhibition potency was ˜60%. Similar results were obtained when the apical and basolateral bathing solutions were switched (high Cl⁻ in apical solution) (FIG. 3B, filled circles). Not wishing to be bound by a particular theory, this suggests that crofelemer inhibition of CFTR does not depend on Cl⁻ concentration. In contrast to the partial inhibition by crofelemer, maximal CFTR inhibition by CFTRᵢₙₕ-172 or GlyH-101 is approximately 100% (see below).

Measurements were done to investigate whether the Crofelemer inhibition potency depended on the CFTR activation mechanism. FIG. 4A shows similar responses to 50 and 500 μM crofelemer using agonists that activate CFTR directly (genistein), or through cAMP-dependent CFTR phosphorylation by increasing cAMP synthesis (forskolin) or reducing cAMP degradation (IBMX). The reversibility of crofelemer inhibition of CFTR was investigated, since washout during secretory diarrhea is a concern in the use of a non-absorbable antisecretory agent. FIG. 4B shows apical current measurements in which CFTR Cl⁻ current was stimulated by CPT-cAMP and then inhibited by different concentrations of crofelemer. Following extensive washing, residual CFTR inhibition was determined from the current after re-stimulation by CPT-cAMP. In control studies in the absence of crofelemer, washout (of CPT-cAMP) followed by re-stimulation produced a similar current to that seen in the initial stimulation. However, following inhibition with different concentrations of crofelemer washout studies showed partial (25-35%) reversal of CFTR inhibition over 30 min. Extended time studies showed <50% reversal of Crofelemer inhibition at 4 h (not shown).

Comparisons of CFTR inhibition in the absence and presence of pre-added crofelemer tested the possibility that the site of action of crofelemer on CFTR might overlap with that of the small-molecule thiazolidinone and glycine hydrazide CFTR inhibitors. FIG. 4C (left) shows concentration-inhibition studies of CFTR inhibition by CFTRᵢₙₕ-172 and GlyH-101. Maximal inhibition ˜100%, with IC₅₀ values of ˜1 and ˜8 μM, respectively. FIG. 4C (right) shows similar concentration-inhibition measurements, in which 50 μM crofelemer was added initially to inhibit CFTR Cl⁻ current by ˜50%. Despite the partial antagonist mechanism of crofelemer, CFTRᵢₙₕ-172 and GlyH-101 inhibited CFTR by nearly 100%. Not wishing to be bound by a particular theory, the similar IC₅₀ values for CFTRᵢₙₕ-172 and GlyH-101 in the absence and presence of crofelemer suggests non-overlapping CFTR inhibition sites for Crofelemer and CFTRᵢₙₕ-172 or GlyH-101.

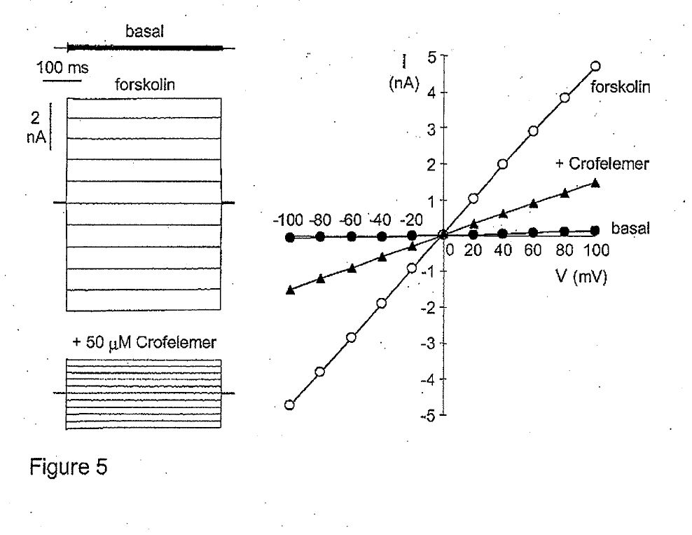

Patch-clamp was done to investigate the molecular mechanism of CFTR inhibition by crofelemer. Whole-cell membrane current was measured in CFTR-expressing FRT cells (FIG. 5, left). Stimulation by 10 μM forskolin produced a membrane current of 179±18 pA/pF (n=3) at +100 mV (total membrane capacitance 15.8±4 pF). Crofelemer at 50 μM gave ˜60% inhibition of CFTR Cl⁻ current. FIG. 5 (right) shows an approximately linear current-voltage relationship for CFTR, as expected for CFTR. While not wishing to be bound by a particular theory, the fact that the CFTR current-voltage relationship remained linear after crofelemer addition suggests a voltage-independent block mechanism, as would be expected for an uncharged inhibitor.

### EXAMPLE 3

### Crofelemer is a Strong Inhibitor of the CaCC TMEM16A

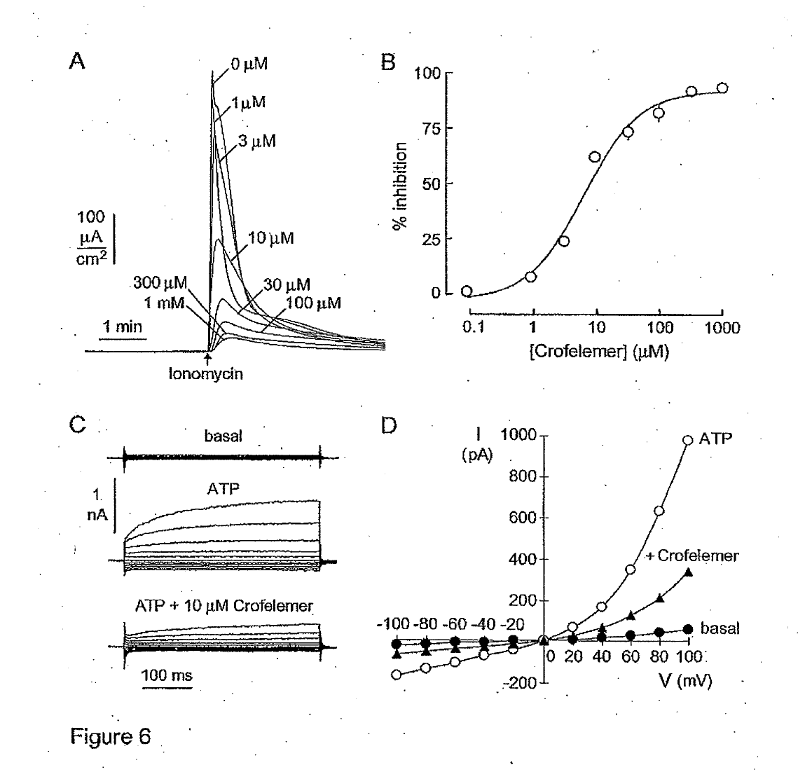

The data in FIG. 2B suggested that crofelemer strongly inhibits CaCC(s) in T84 cells. To test whether the protein TMEM16A is the CaCC target of crofelemer, FRT epithelial cells stably expressing TMEM16A were pretreated with different concentrations of Crofelemer, followed by addition of 1 μM ionomycin to stimulate TMEM16A Cl⁻ current. Measurements were made in the presence of a transepithelial Cl⁻ gradient, so that current is a direct, quantitative measure of TMEM16A Cl⁻ conductance. FIG. 6A shows crofelemer concentration-dependent inhibition of TMEM16A Cl⁻ current, which was nearly complete at high concentrations of crofelemer. FIG. 6B shows an IC₅₀ for Crofelemer inhibition of TMEM16A of ˜6.5 μM.

Whole-cell membrane current was measured in TMEM16A-expressing FRT cells (FIG. 6C). Stimulation by 100 μM ATP produced a membrane current of 56±13 pA/pF (n=3) at +100 mV. Pretreatment with 10 μM crofelemer inhibited ATP-induced TMEM16A Cl⁻ current by 58% (24±6 pA/pF, n=3). FIG. 6D shows an outward rectifying current-voltage relationship for TMEM16A. The TMEM16A current-voltage relationship remained outward rectifying after crofelemer addition, as expected for an uncharged inhibitor. These results suggest that there is at least a second, distinct luminal membrane Cl⁻ channel target of Crofelemer.

### EXAMPLE 4

### Crofelemer has Little Effect on Apical Cation Channels and Camp/Calcium Signaling

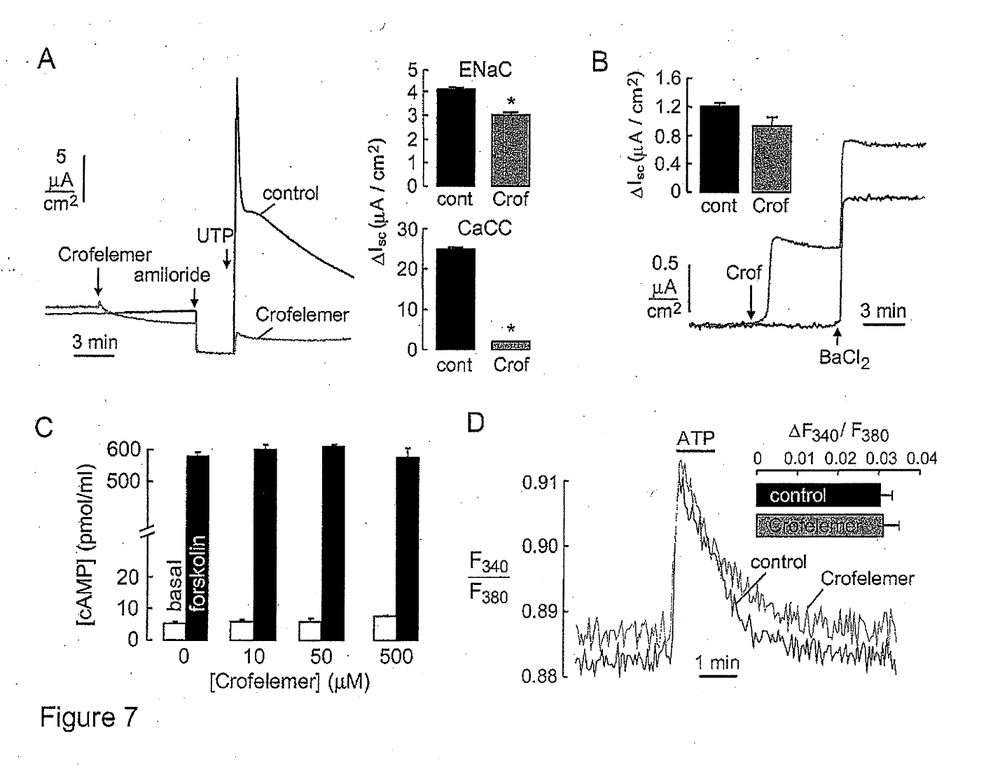

The apical membrane of enterocytes also contains Na⁺ and K⁺ channels, which are also potential targets of crofelemer. To investigate whether crofelemer alters the activity of the epithelial cell Na⁺ channel ENaC, short-circuit current was measured in primary cultures of human bronchial epithelial cells, which robustly express ENaC and in which the change in short-circuit current following amiloride provides a quantitative measure of ENaC activity (Yamaya et al., 1994). FIG. 7A shows that pre-treatment of the cell culture with 50 μM crofelemer produced a small, ˜20% inhibition of ENaC activity. Human bronchial epithelial cells also express TMEM16A and have robust CaCC activity. Crofelemer pre-treatment produced a >90% reduction in short-circuit current following the calcium-elevating agonist UTP, consistent with the results in T84 cells and TMEM16A-transfected FRT cells, above.

Possible inhibition of apical K⁺ channels by crofelemer was tested in human bronchial epithelial cells in which the basolateral membrane was permeabilized with amphotericin B in the presence of a transepithelial K⁺ gradient. Under these conditions, the small measured current is an apical membrane K⁺ current. Apical K⁺ current was measured following addition of BaCl₂, a nonspecific inhibitor of K⁺ channels. FIG. 7B shows that pre-treatment with 50 μM crofelemer produced a small, ˜22% inhibition of apical membrane K⁺ current.

The possibility that crofelemer action on apical membrane receptor(s) might affect major intracellular signaling pathways, which might secondarily modulate the activities of basolateral membrane transporters to inhibit transcellular Cl⁻ secretion indirectly was tested. In FIG. 7C, crofelemer at 50 μM had no significant effect on basal or forskolin-stimulated cAMP concentrations in T84 cells. In FIG. 7D, crofelemer did not alter basal cytoplasmic calcium concentration, nor did it affect the elevation in calcium concentration following ATP treatment in T84 cells.

Examples of some reagents and protocols that can be used in the above examples include but are not limited to the following: forskolin, apigenin and 3-isobutyl-1-methylxanthine (IBMX) were purchased from Sigma. 8-(4-chlorophenylthio)-cAMP (CPT-cAMP) was purchased from Calbiochem. The small-molecule CFTR inhibitors CFTRᵢₙₕ-172 and GlyH-101, and the CaCC inhibitor CaCCᵢₙₕ-01, were synthesized as reported (Ma et al., 2002; Muanprasat et al., 2004; de la Fuente et al., 2008). Crofelemer was provided by Napo Pharmaceuticals Inc. (South San Francisco, Calif.). Crofelemer was prepared by extraction from the bark latex of C. lechleri. After chilling the bark latex to induce a phase separation, the solid residues were discarded and the supernatant was extracted with butanol. The crofelemer-containing aqueous phase was filtered by tangential flow and subjected to low pressure liquid chromatography on an ion exchange column. The crofelemer-enriched fraction was purified on a Sephadex column, with crofelemer eluted using a mobile phase of aqueous acetone. Crofelemer was then dried under vacuum.

Crofelemer consists of a mixture of proanthocyanidin oligomers with an average molecular weight of 2100 daltons, in agreement with previously reported average molecular weight of 2300 daltons (Ubillas et al., 1994). FIG. 2A shows the structure of crofelemer. The material used for the studies here is the same as that used in clinical trials, where it is formulated for oral dosing as modified-release tablets (125 or 250 mg crofelemer per tablet).

Fisher rat thyroid (FRT) cells expressing human CFTR were generated as described (Ma et al., 2002). FRT cells expressing human TMEM16A (cDNA provided by Dr. Luis Galietta, Gaslini Institute, Genoa, Italy) were generated similarly. FRT cells were cultured in F-12 Modified Coon's Medium (Sigma) supplemented with 10% fetal bovine serum (Hyclone), 2 mM glutamine, 100 units/ml penicillin, 100 μg/ml streptomycin, 350 μg/ml hygromycin and 500 μg/ml geneticin. Primary cultures of human bronchial epithelial cells were maintained at an air-liquid interface as described (Yamaya et al., 1992). T84 cells were cultured in DMEM/Ham's F-12 (1:1) medium containing 10% FBS, 100 units/ml penicillin and 100 μg/ml streptomycin. Cells were grown on Snapwell porous filters (Costar 3801) at 37° C. in 5% CO₂/95% air.

FRT cells (stably expressing CFTR or TMEM16A) were cultured on Snapwell filters until confluence (transepithelial resistance >500 ohm.cm). Short-circuit current was measured in Ussing chambers (Vertical diffusion chamber; Costar) with Ringer's solution bathing the basolateral surface and half-Ringer's bathing the apical surface. Ringer's solution contained: 130 mM NaCl, 2.7 mM KCl, 1.5 mM KH2PO4, 1 mM CaCl2, 0.5 mM MgCl2, 10 mM Na-HEPES, 10 mM glucose, pH 7.3. Half-Ringer's solution was the same, except that 65 mM NaCl was replaced with Na gluconate, and CaCl2 was increased to 2 mM. The basolateral membrane was permeabilized with 250 μg/ml amphotericin B, as described (Ma et al., 2002). Chambers were bubbled continuously with air. For T84 cells and bronchial epithelial cells, cells were bathed in symmetrical HCO3—buffered solution containing (in mM): 120 NaCl, 5 KCl, 1 MgCl2, 1 CaCl2, 10 D-glucose, 5 HEPES, and 25 NaHCO3 (pH 7.4), and aerated with 5% CO2 at 37° C. For the measurement of apical K+ conductance in T84 cells, NaHCO3 and NaCl were replaced with Na gluconate, and Na gluconate in basolateral solution was replaced with K gluconate and bubbled with air. The basolateral membrane was permeabilized with 20 μM amphotericin B. Short-circuit current was measured using a DVC-1000 voltage-clamp apparatus (World Precision Instruments).

T84 cells were grown in 24-well plates, treated for 45 min with crofelemer, then for 10 min with 0 or 20 μM forskolin, lysed by sonication, centrifuged to remove cell debris, and the supernatant was assayed for cAMP according to manufacturer's instructions (Parameter™ cAMP immunoassay kit, R&D Systems).

Whole-cell recordings were made on FRT cells stably expressing CFTR or TMEM16A. The pipette solution for CFTR contained 140 mM N-methyl D-glucamine chloride (NMDG-Cl), 5 mM EGTA, 1 mM MgCl2, 1 mM Tris-ATP and 10 mM HEPES (pH 7.2). The pipette solution for TMEM16A contained 130 mM CsCl, 0.5 mM EGTA, 1 mM MgCl2, 1 mM Tris-ATP and 10 mM HEPES (pH 7.2). The bath solution contained 140 mM N-methyl D-glucamine chloride, 1 mM CaCl2, 1 mM MgCl2, 10 mM glucose and 10 mM HEPES (pH 7.4). All measurements were done at room temperature (22-25° C.). Pipettes were pulled from borosilicate glass and had resistances of 3-5 Mohm after fire polishing. Seal resistances were between 3 and 10 Gohm. After establishing the whole-cell configuration, CFTR was activated by forskolin and IBMX, and TMEM16A by ATP. Whole-cell currents were elicited by applying hyperpolarizing and depolarizing voltage pulses from a holding potential of 0 mV to potentials between −100 mV and +100 mV in steps of 20 mV. The current output was filtered at 5 kHz. Currents were digitized and analyzed using an AxoScope 10.0 system and a Digidata 1440A AC/DC converter.

Measurements of [Ca2+]i in confluent monolayers of T84 cells were done by loading cells with fura-2 by 30 min incubation at 37° C. with 2 μM fura-2-AM (Molecular Probes). Fura-2 loaded T84 cells were mounted in a perfusion chamber on the stage of an inverted fluorescence microscope. The cells were superfused with (in mM): 140 NaCl, 5 KCl, 1 MgCl2, 1 CaCl2, 10 D-glucose and 10 HEPES (pH 7.4). Fura-2 fluorescence was recorded at excitation wavelengths of 340 nm and 380 nm and the results were expressed as a 340/380 fluorescence ratio. After obtaining baseline measurements, 100 μM ATP was added in the perfusate. Measurements were made in the absence and presence of 50 μM Crofelemer.

### EXAMPLE 5

### Method of Reducing the Symptoms of Diarrhea, Secretory Diarrhea, Irritable Bowel Syndrome, Constipation, and Crohn's Disease in a Human Patient

A human patient suffering from diarrhea, secretory diarrhea, irritable bowel syndrome, constipation, or Crohn's disease, is identified. A dosage of, for example, 4 mg/kg of crofelemer is administered orally, twice daily, to the patient. The dosage can be adjusted so that it is enough to be effective in reducing abnormal stool weight and frequency of elimination.

### EXAMPLE 6

### Method of Reducing the Symptoms of AIDS-Associated Diarrhea in a Human Patient

A human patient suffering from AIDS-associated diarrhea is identified. A dosage of, for example, 4 mg/kg of crofelemer is administered orally, twice daily, to the patient. The dosage can be adjusted so that it is enough to be effective in reducing abnormal stool weight and frequency of elimination.

### EXAMPLE7

### Method of Reducing the Symptoms of Irritable Bowel Syndrome in a Human Patient

A human patient suffering from irritable bowel syndrome is identified. A dosage of, for example, 4 mg/kg of crofelemer is administered orally, twice daily, to the patient. The dosage can be adjusted so that it is enough to be effective in reducing abnormal stool weight, frequency of elimination and/or pain. Treatment is considered successful if the number of pain-free days is increased.

### EXAMPLE 8

### Method of Reducing the Symptoms of Cholera in a Human Patient

A human patient suffering from Cholera is identified. Additionally, a dosage of, for example, 4 mg/kg of crofelemer is administered orally, twice daily, to the patient. The dosage can be adjusted so that it is enough to be effective in reducing abnormal stool weight and frequency of elimination.

### EXAMPLE 9

### Method of Reducing the Symptoms of Cholera in a Human Patient with a Combination of Antibiotic and Crofelemer

A human patient suffering from Cholera is identified. An effective dose of azithromycin is administered to the patient. Additionally, a dosage of, for example, 4 mg/kg of crofelemer is administered orally, twice daily, to the patient. The dosage can be adjusted so that it is enough to be effective in reducing abnormal stool weight and frequency of elimination.

### EXAMPLE 10

### Method of Reducing the Symptoms of Cholera in a Human Patient with a Combination of Rehydration Therapy and Crofelemer

A human patient suffering from cholera is identified. An Oral Rehydration Salts (ORS) solution containing specific proportions of water, salts, and sugar is administer to the patient. A dosage of, for example, 4 mg/kg of crofelemer is administered orally, twice daily, to the patient. The dosage can be adjusted so that it is enough to be effective in reducing abnormal stool weight and frequency of elimination.

### EXAMPLE 11

### Method of Reducing the Symptoms of Diarrhea, Secretory Diarrhea, Irritable Bowel Syndrome, Constipation, and Crohn's Disease in a Human Patient by Intravenous Administration of Crofelemer

A human patient suffering from diarrhea, secretory diarrhea, irritable bowel syndrome, constipation, or Crohn's disease, is identified. A dosage of, for example, 4 mg/kg of crofelemer is administered intravenously to the patient. The dosage can be adjusted so that it is enough to be effective in reducing abnormal stool weight and frequency of elimination.

### EXAMPLE 12

### Method of Reducing the Symptoms of Diarrhea, Secretory Diarrhea, Irritable Bowel Syndrome, Constipation, and Crohn's Disease in a Human Patient by Administration of Crofelemer and Thiazolidinone

A human patient suffering from diarrhea, secretory diarrhea, irritable bowel syndrome, constipation, or Crohn's disease, is identified. A dosage of, for example, 4 mg/kg of crofelemer in combination with an effective amount of thiazolidinone is administered intravenously to the patient. The dosage can be adjusted so that it is enough to be effective in reducing abnormal stool weight and frequency of elimination.

### EXAMPLE 13

### Method of Reducing the Symptoms of Diarrhea, Secretory Diarrhea, Irritable Bowel Syndrome, Constipation, and Crohn's Disease in a Human Patient by Administration of Crofelemer and Glycine Hydrazide

A human patient suffering from diarrhea, secretory diarrhea, irritable bowel syndrome, constipation, or Crohn's disease, is identified. A dosage of, for example, 4 mg/kg of crofelemer in combination with an effective amount of glycine hydrazide is administered intravenously to the patient. The dosage can be adjusted so that it is enough to be effective in reducing abnormal stool weight and frequency of elimination.

### EXAMPLE 14

### Method of Reducing the Symptoms of Diarrhea, Secretory Diarrhea, Irritable Bowel Syndrome, Constipation, and Crohn's Disease in a Human Patient

A human patient suffering from diarrhea, secretory diarrhea, irritable bowel syndrome, constipation, or Crohn's disease, is identified. A dosage of, for example, 7 mg/kg of crofelemer is administered orally, twice daily, to the patient. The dosage can be adjusted so that it is enough to be effective in reducing abnormal stool weight and frequency of elimination.

### EXAMPLE 15

### Method of Reducing the Symptoms of AIDS-Associated Diarrhea in a Human Patient

A human patient suffering from AIDS-associated diarrhea is identified. A dosage of, for example, 7 mg/kg of crofelemer is administered orally, twice daily, to the patient. The dosage can be adjusted so that it is enough to be effective in reducing abnormal stool weight and frequency of elimination.

### EXAMPLE 16

### Method of Reducing the Symptoms of Irritable Bowel Syndrome in a Human Patient

A human patient suffering from irritable bowel syndrome is identified. A dosage of, for example, 7 mg/kg of crofelemer is administered orally, twice daily, to the patient. The dosage can be adjusted so that it is enough to be effective in reducing abnormal stool weight, frequency of elimination and/or pain. Treatment is considered successful if the number of pain-free days is increased.

### EXAMPLE 17

### Method of Reducing the Symptoms of Cholera in a Human Patient

A human patient suffering from Cholera is identified. Additionally, a dosage of, for example, 7 mg/kg of crofelemer is administered orally, twice daily, to the patient. The dosage can be adjusted so that it is enough to be effective in reducing abnormal stool weight and frequency of elimination.

### EXAMPLE 18

### Method of Reducing the Symptoms of Cholera in a Human Patient With a Combination of Antibiotic and Crofelemer

A human patient suffering from Cholera is identified. An effective dose of azithromycin is administered to the patient. Additionally, a dosage of, for example, 7 mg/kg of crofelemer is administered orally, twice daily, to the patient. The dosage can be adjusted so that it is enough to be effective in reducing abnormal stool weight and frequency of elimination.

### EXAMPLE 19

### Method of Reducing the Symptoms of Cholera in a Human Patient With a Combination of Rehydration Therapy and Crofelemer

A human patient suffering from cholera is identified. An Oral Rehydration Salts (ORS) solution containing specific proportions of water, salts, and sugar is administer to the patient. A dosage of, for example, 7 mg/kg of crofelemer is administered orally, twice daily, to the patient. The dosage can be adjusted so that it is enough to be effective in reducing abnormal stool weight and frequency of elimination.

### EXAMPLE 20

### Method of Reducing the Symptoms of Diarrhea, Secretory Diarrhea, Irritable Bowel Syndrome, Constipation, and Crohn's Disease in a Human Patient by Intravenous Administration of Crofelemer

A human patient suffering from diarrhea, secretory diarrhea, irritable bowel syndrome, constipation, or Crohn's disease, is identified. A dosage of, for example, 7 mg/kg of crofelemer is administered intravenously to the patient. The dosage can be adjusted so that it is enough to be effective in reducing abnormal stool weight and frequency of elimination.

### EXAMPLE 21

### Method of Reducing the Symptoms of Diarrhea, Secretory Diarrhea, Irritable Bowel Syndrome, Constipation, and Crohn's Disease in a Human Patient by Administration of Crofelemer and Thiazolidinone

A human patient suffering from diarrhea, secretory diarrhea, irritable bowel syndrome, constipation, or Crohn's disease, is identified. A dosage of, for example, 7 mg/kg of crofelemer in combination with an effective amount of thiazolidinone is administered intravenously to the patient. The dosage can be adjusted so that it is enough to be effective in reducing abnormal stool weight and frequency of elimination.

### EXAMPLE 22

### Method of Reducing the Symptoms of Diarrhea, Secretory Diarrhea, Irritable Bowel Syndrome, Constipation, AND Crohn's Disease IN A HUMAN PATIENT by Administration of Crofelemer and Glycine Hydrazide

A human patient suffering from diarrhea, secretory diarrhea, irritable bowel syndrome, constipation, or Crohn's disease, is identified. A dosage of, for example, 7 mg/kg of crofelemer in combination with an effective amount of glycine hydrazide is administered intravenously to the patient. The dosage can be adjusted so that it is enough to be effective in reducing abnormal stool weight and frequency of elimination.

### EXAMPLE 23

### Method of Reducing the Symptoms of Cholera in a Human Patient

A human patient suffering from Cholera is identified. Additionally, a dosage of, for example, 4 mg/kg of crofelemer is administered intravenously, twice daily, to the patient. The dosage can be adjusted so that it is enough to be effective in reducing abnormal stool weight and frequency of elimination.

### EXAMPLE24

### Method of Reducing the Symptoms of Cholera in a Human Patient

A human patient suffering from Cholera is identified. Additionally, a dosage of, for example, 7 mg/kg of crofelemer is administered orally, twice daily, to the patient. The dosage can be adjusted so that it is enough to be effective in reducing abnormal stool weight and frequency of elimination.

What is claimed is:

1. A method of modulating Cl⁻ secretion in a cell, comprising contacting a cell comprising a calcium-activated chloride channel (CaCC) with an effective amount of crofelemer.

2. The method of claim 1 wherein the CaCC is TMEM16A CaCC.

3. A The method of claim 1, wherein the cell further comprises one or more of a cystic fibrosis transmembrane conductance regulator (CFTR) and an epithelial sodium channel (ENaC).

4. A method of modulating Na⁺ secretion of a cell, comprising contacting a cell comprising a Na⁺ channel with an effective amount of crofelemer.

5. The method of claim 4 wherein the Na⁺ channel is epithelial sodium channel (ENaC).

6-32. (canceled)

33. The method of claim 1, wherein contacting the cell comprises contacting an intestinal cell or a bronchial cell.

34. The method of claim 1, wherein the effective amount of crofelemer is about 1-1,000 μM.

35. The method of claim 1, wherein the effective amount of crofelemer comprises from about 10 to about 2,000 mg of crofelemer.

36. The method of claim 4, wherein the effective amount of crofelemer comprises from about 10 to about 2,000 mg of crofelemer.

37. A method of treating a patient suffering from a calcium-activated chloride channel (CaCC) chanelpathy disorder comprising administering an effective amount of a pharmaceutical composition comprising crofelemer to the patient.

38. The method of claim 37, wherein the CaCC channelpathy disorder is selected from a group consisting of Cystic fibrosis, Erythromelalgia, Hyperkalemic periodic paralysis, Hypokalemic periodic paralysis, Long QT syndrome, Short QT syndrome, Malignant hyperthermia, Myotonia cogenita, and Neuromytonia.

39. The method of claim 37, wherein the pharmaceutical composition comprises from about 10 to about 2,000 mg crofelemer.
